(19) United States
(12) Patent Application Publication
(10) Pub. No.: US 2020/0173994 A1
(43) Pub. Date: Jun. 4, 2020

(54) METHOD FOR EVALUATING SEVERITY OF DENGUE VIRUS INFECTION IN INDIVIDUAL, DETECTION DEVICE AND DETECTION

(71) Applicant: Tzong-Shiann HO, Tainan City (TW)

(72) Inventors: Tzong-Shiann HO, Tainan City (TW); Yung-Chun CHUANG, Hsinchu City (TW); Po-Chih HSU, Tainan City (TW); Yu-Wei CHENG, Tainan City (TW); Ching-Chuang LIU, Tainan City (TW); Trai-Ming YEH, Tainan City (TW)

(21) Appl. No.: 16/620,829

(22) PCT Filed: Jun. 9, 2017
(86) PCT No.: PCT/CN2017/087750

§ 371 Date: Dec. 9, 2019

(51) Int. Cl. G01N 33/569; G01N 33/573

(52) U.S. Cl. CPC G01N 33/56983; G01N 2333/974; G01N 33/573

(57) ABSTRACT

Provided is a method for evaluating the severity of the dengue virus infection in an individual, executed by means of detecting a biological sample of an individual in vitro. The method comprises: detecting whether a non-structural protein 1 is present in a biological sample in vitro to obtain a first detection result; detecting whether a complex is present in the biological sample in vitro to obtain a second detection result, wherein the complex comprises the non-structural protein 1 and a thrombin, or comprises the non-structural protein 1 and a prothrombin; and evaluating the severity of the dengue virus infection in the individual via the first detection result and the second detection result. Also provided are a detection device and a detection kit for evaluating the severity of the dengue virus infection in an individual.

### FIELD OF THE INVENTION

The present invention relates to a method for evaluating severity of dengue virus infection in an individual. The present invention also relates to a detection device and a detection kit for evaluating the severity of the dengue virus infection in an individual.

### BACKGROUND OF THE INVENTION

Dengue fever is a mosquito-borne tropical disease caused by the infection of dengue virus (DENY). According to the Handbook for Clinical Management of Dengue released by World Health Organization (WHO), patients infected with dengue viruses can be divided into the following three groups depending on their severity: (1) the group A patient is characterized by without warning signs, such as abdominal pain or tenderness, persistent vomiting, clinical fluid accumulation or mucosal bleed; (2) the group B patient is characterized by the presence of the above-mentioned warning signs; and (3) the group C patient is characterized by the presence of shock and fluid accumulation with respiratory distress caused by severe plasma leakage, severe bleeding or severe organ impairment.

Dengue virus is a single positive-stranded RNA virus of Flaviviridae (Flavivirus), which can be mainly divided into four serotypes including DENY-1, DENV-2, DENY-3 and DENV-4 depending on the different antigenicity. Symptoms such as mouth and nose bleeding, headache, vomiting, rash, muscle and joints pain appears in the individual infected with dengue viruses. The severe cases may even occur abnormal hemostasis, dengue hemorrhagic fever (DHF) and dengue shock syndrome (DSS). Therefore, preventing and controlling dengue fever have become a subject valued by researchers in the art.

The genome of dengue virus comprises about 11,000 bases which can encode three structural proteins, that is, capsid protein C, membrane protein M and envelope protein E, and seven non-structural proteins (NS), that is, non-structural protein 1 (NS1), non-structural protein 2A (NS2A), non-structural protein 2B (NS2B), non-structural protein 3, (NS3), non-structural protein 4A (NS4A), non-structural protein 4B (NS4B) and non-structural protein 5 (NS5), wherein non-structural proteins are found merely in the infected host cells. Studies have pointed out that non-structural protein 1 (NS1) plays an important role in the replication of dengue virus and is closely associated with symptoms caused by dengue fever, such as dengue hemorrhagic fever (see, Libraty D. H. et al. (2002), J. Infect. Dis., 186:1165-1168; and Fan J. et al. (2014), Virol. Sin., 29:162-169).

NS1 is a glycoprotein having 46 to 50 kDa, which is expressed in the cells of infected mammal and divided into secreted and membrane-associated forms. Owing to the close relation between NS1 and dengue fever, NS1 has been used as an important marker for detecting whether an individual is infected with dengue viruses by the researchers. For example, a literature reported that the detection of NS1 is important for early diagnosis of dengue virus infection (see, Badave G. K. et al. (2015), Int. J. Curr Microbiol. App. Sci., 4:779-784).

Currently, the known methods for detecting dengue virus infection are mainly using anti-NS1 specific antibodies alone and detecting them by immunological methods. However, in the known methods, no matter what the viruses are isolated from the patient or the type of virus is determined by reverse transcription-polymerase chain reaction (RT-PCR), they are extremely time-consuming, costly and heavily laboratory equipment-depended detection methods, and have a narrow range of applications.

In addition, the known detection methods must be operated under the rigorous conditions to avoid affecting the detection results due to the accidental contamination. More importantly, NS1 as a detection marker alone is insufficient in sensitivity, and often causes the misdiagnosis or delayed treatment.

Moreover, some studies utilized the antibodies specific binding with a complex containing NS1 and thrombin or with a complex containing NS1 and prothrombin to detect the dengue virus infection. For example, TW I428598 B disclosed a method for detecting Flaviviridae virus infection by way of detecting whether a biological sample contains a complex formed by two proteins of NS1 and thrombin or two proteins of NS1 and prothrombin in order to determine whether the patient is infected with dengue viruses. US 2016/0363590 A1 disclosed a method for early prediction of severe dengue fever or hemorrhagic dengue by using olfactomedin 4 or NS1 as a protein marker.

In the above-mentioned prior technologies, whether an individual is infected with dengue viruses is determined by detecting whether a complex containing NS1 and thrombin or a complex containing NS1 and prothrombin exists in an in vitro biological sample. However, there are considerable differences in the severity of dengue fever, and the most severe cases may cause death possibly. If the patients are only aware that they infect with dengue viruses but fail further to understand the severity of the infection, it is likely that the conditions will be ignored and the treatment will be delayed. Therefore, if a method, detection device and detection kit for evaluating severity of dengue virus infection in individual by detecting an in vitro biological samples can be developed, it will make a breakthrough in the treatment strategy of dengue fever.

### SUMMARY OF THE INVENTION

In view of the conventional methods or technologies, whether an individual is infected with dengue viruses is determined only by detecting whether a complex containing NS1 and thrombin or a complex containing NS1 and prothrombin exists in an in vitro biological sample, the severity of dengue virus infection in a patient cannot be identified quickly, and causes the patient to ignore the conditions and even to delay the timing of treatment.

In the present invention, the inventors discovered and demonstrated that the method according to the present invention can accurately detect the dengue virus infection in an individual with high sensitivity and high accuracy. When both NS1 and a complex containing NS1 and thrombin are present in an in vitro biological sample, it indicates that the individual has a more serious dengue infection.

The above-mentioned and other objects, features and outstanding effects of the present invention will be described in the following paragraphs.

The present invention provides a method for evaluating severity of dengue virus infection in an individual, which is executed by means of detecting an in vitro biological sample of the individual. The method comprises the following steps: detecting whether NS1 is present in an in vitro biological sample to obtain a first detection result; detecting whether complex is present in the in vitro biological sample to obtain a second detection result, wherein said complex contains NS1 and thrombin or NS1 and prothrombin; and evaluating the severity of the dengue virus infection in the individual via the first detection result and the second detection result.

The present invention also provides a detection device for evaluating severity of dengue virus infection in an individual. The detection device comprises a first detection unit and a second detection unit. The first detection unit is used for detecting whether NS1 is present in an in vitro biological sample to obtain a first detection result. The second detection unit is used for detecting whether a complex is present in the in vitro biological sample to obtain a second detection result, wherein said complex contains NS1 and thrombin or NS1 and prothrombin. Then, the severity of the dengue virus infection in the individual is evaluated via the first detection result and the second detection result.

The present invention also provides a detection kit comprising the above-mentioned detection device.

As used herein, the terms “dengue virus”, “dengue fever virus” and “DENV” can be used interchangeably.

As used herein, the term “severity” means the level or intensity of distressing condition, symptom, or a group of symptoms.

As used herein, the term “biological sample” means all substances produced by or separable from an organism.

In one embodiment, the biological sample includes one of followings: blood (such as serum, plasma or whole blood), urine, saliva and lymph. In a preferred embodiment, the biological sample is serum.

In one embodiment, the individual belongs to a group B patient with dengue virus infection or a group C patient with dengue virus infection if one of complex containing NS1 and thrombin and complex containing NS1 and prothrombin as well as NS1 are simultaneously present in the biological sample.

The above-mentioned “group B patient with dengue virus infection” means a dengue patient with warning signs, such as abdominal pain or tenderness, persistent vomiting, clinical fluid accumulation, and mucosal bleed. The Handbook for Clinical Management of Dengue released by World Health Organization (WHO) can be referred.

The above-mentioned “group C patient with dengue virus infection” means a dengue patient with shock and fluid accumulation with respiratory distress caused by severe plasma leakage, severe bleeding, or severe organ impairment. The Handbook for Clinical Management of Dengue released by World Health Organization (WHO) can be referred.

In one embodiment, the step of detecting whether NS1 is present in the in vitro biological sample is carried out by using an antibody specifically recognizing NS1.

In one embodiment, the step of detecting whether the complex is present in the in vitro biological sample is carried out by using an antibody specifically recognizing the complex.

In one embodiment, the antibody is a monoclonal antibody or a polyclonal antibody. In the preferred embodiment, the antibody specifically recognizing NS1 is a monoclonal antibody. In another preferred embodiment, the antibody specifically recognizing the complex is a polyclonal antibody.

The above-mentioned “antibody” includes “an antibody-based binding moiety”, “immunoglobulin molecules”, and “immunologically active determinants”, such as a molecular having an antigen-binding site specifically binding (immune response) to NS1 or the above-mentioned complex, and intends to cover all isotypes of antibody, such as IgG, IgA, IgM and IgE, and the fragments specifically binding to NS1 or the above-mentioned complex.

The above-mentioned “antibody” also includes a capture antibody and a detection antibody.

The above-mentioned “capture antibody” means an antibody, whether it is a monoclonal antibody, a polyclonal antibody, or immunoreactive fragments of an antibody, which is capable of binding to an antigen of interest and thus allows to identify said antigen by a subsequently applied antibody. The capture antibody can be used in a heterogeneous (solid phase) or homogeneous (solution phase) assay. Preferably, the capture antibody is fixed on a solid phase, such as a substrate manufactured by nylon, polystyrene, polyvinylchloride, nitrocellulose, polyvinylidene fluoride (PVDF) and the similar materials. The substrates can have various forms, such as hole plate, column or test paper. The capture antibody can also bind to the antigen of the biological sample in a suspension, depending on the subsequent method for separating the capture antibody that it bound to the antigen and the capture antibody that it unbound to the antigen, and on whether the user is convenient or not in operation. The invention is not limited herein.

The above-mentioned “detection antibody” means a antibody comprising a detectable label, and the detectable label is specific for one or more analytes of interest in the sample. Antibodies specific for one or more analytes of interest are also included, wherein the antibodies can bind to another species comprising a detectable label. The examples of detectable labels include, but are not limited to, a hapten label, such as biotin/streptavidin and digoxigenin (Dig); a nucleic acid label, such as oligonucleotide label, a chemiluminescent label; a fluorescent label, such as FITC (fluorescein isothiocynate), 5-carboxyfluorescein, 6-carboxyfluorescein, anthocyanin, phycoerythrin and rhodamine; an enzymatic label, such as luciferase, alkaline phosphatase, catalase, horseradish peroxidase (HRP), β-galactosidase, uricase and lactoperoxidase; a radioactive label, such as H³, C¹¹, C¹⁴, P³², s³⁵, I¹²³, I¹²⁴, I¹²⁵, I¹³¹, Tc¹¹¹ and Lu¹⁷⁷; an epitope tag, such as T7, c-Myc, HA, VSV-G, HSV, FLAG, V5 and HIS; and the combination thereof.

In one embodiment, the individual is a mammal. In the following examples, the human is used as a representative.

In one embodiment, the detection is carried out by using any one of following methodologies: enzyme linked immunosorbent assay (ELISA), dot blotting, lateral flow assay (LFA), multiplex immunoassay, radioimmunoassay (RTA), immunoradiometric assay (IRMA), fluorescent immunoassay (FIA), chemiluminescent immunoassay and immunonephelometry.

In one embodiment, the dengue viruses include various serotypes, such as DENV-1, DENV-2, DENY-3 and DENV-4.

In one embodiment, the organisms for the biological sample include animal hosts that can be infected with dengue viruses. Preferably, the organisms are mammals, such as human, monkey, rat, cattle, ovis, canine, feline and porcine. In the following examples, the organism is a human.

In one embodiment, the biological sample may have any form according to the main extent within an individual affected by the dengue viruses, and include blood (such as serum, plasma or whole blood), urine, saliva, lymph, and nearby tissues or cells through which the blood, urine or lymph flows, and the like. Preferably, the biological sample contains cells, such as nerve cells, muscle cells, liver cells, endothelial cells, blood cells and lymphocytes, may be infected by dengue viruses. More preferably, the biological sample contains mammalian endothelial cells or blood cells. The biological sample can be a fresh, tissue cultured or refrigerated or frozen sample. Moreover, the biological sample can be a sample that further raises the antigen concentration by the purification, centrifugation, extraction or concentration process.

Summing up the above, a method for evaluating severity of dengue virus infection in an individual, detection device and detection kit provided by the present invention, in addition to being able to effectively detect whether an organism is infected by dengue viruses, more importantly, can provide higher sensitivity and accuracy comparing with the known technologies.

All patents and literatures cited in the present specification are hereby incorporated herein as a part of the specification by reference.

### BRIEF DESCRIPTION OF THE DRAWINGS

FIG. 1 shows a flowchart of a method for evaluating the severity of the dengue virus infection in an individual according to the present invention; and

FIG. 2 shows a schematic diagram of a preferred embodiment according to the present invention.

### DETAILED DESCRIPTION OF THE INVENTION

The present invention is further illustrated by the following embodiments and examples. However, it should be understood that the embodiments and examples are only for illustrative purposes and in no way a limitation of the present invention.

### Embodiments

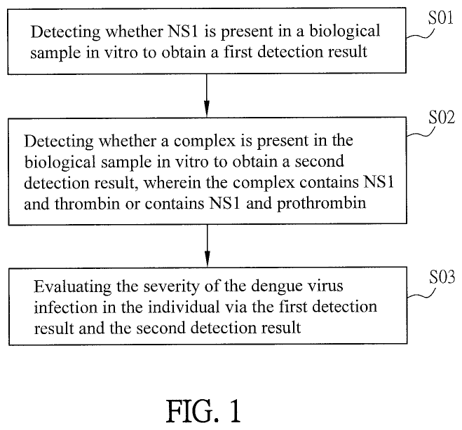

An embodiment of the present invention will be described below with reference to the related drawings. The embodiment describes a method for evaluating the severity of the dengue virus infection in an individual with high sensitivity and high accuracy. Said method executed by means of detecting an in vitro biological sample of an individual. The flowchart of said method is shown in FIG. 1, and the steps comprise: detecting whether a non-structural protein 1 (NS1) is present in an in vitro biological sample to obtain a first detection result (Step S01); detecting whether a complex is present in the in vitro biological sample to obtain a second detection result, wherein the complex contains NS1 and thrombin or NS1 and prothrombin (Step S02); and evaluating the severity of the dengue virus infection in the individual via the first detection result and the second detection result (Step S03).

In the Step S01 of this embodiment, the in vitro biological sample is obtained from the individual with or suspected of having dengue virus infection. In the examples of the present invention, the individual is a human. In the examples of the present invention, the biological sample is a serum.

In the Step S02 of this embodiment, NS1 and thrombin or NS1 and prothrombin conjugated mutually. That is, both NS1 and thrombin or both NS1 and prothrombin have positions capable of interaction or covalent bonding. In the embodiment of the present invention, NS1 and thrombin or NS1 and prothrombin can also indirectly conjugate via other molecules. In the embodiment of the present invention, the serum has multiple complexes, and the partial complexes contain NS1 and thrombin and the partial complexes contain NS1 and prothrombin. In the embodiment of the present invention, the complexes can simultaneously contain NS1 and thrombin as well as NS1 and prothrombin, and the numbers of NS1, thrombin and prothrombin are not limited. The mutual conjugating relation may also be the mutual conjugation of one NS1 and two thrombin or prothrombin, or the mutual chimerism of two or more NS1 and one thrombin or one prothrombin. The invention is not limited herein. In an example of the present invention, the form of NS1 include, but are not limited to, a secreted protein secreted to out of cell, and a membrane-associated protein binding on the surface of host cell. In another example of the present invention, NS1 can be in the form without post-translational modification, or in the form of post-translational modification, such as glycosyation, phosphorylation and the like. In an example of the present invention, the thrombin or prothrombin can be isolated or purified from an organism, or be a functional protein made by artificial synthesis. These separation, purification and artificial synthesis techniques can be understood by a person ordinarily skilled in the art.

In the Step S03 of this embodiment, if the first detection result is that NS1 is present in the in vitro biological sample as well as the second detection result is that the complex is present in the in vitro biological sample, that is, if one of the complex containing NS1 and thrombin and the complex containing NS1 and prothrombin as well as NS1 are simultaneously present in the biological sample, it indicates that the individual belongs to a group B patient with dengue virus infection or a group C patient with dengue virus infection. In an example of the present invention, the first detection result is that NS1 is present in the in vitro biological sample, and the second detection result is that the complex containing NS1 and thrombin is present in the in vitro biological sample.

The present invention also provides another embodiment, which relates to a detection device. The detection device is used for evaluating the severity of the dengue virus infection in an individual. The detection device comprises a first detection unit and a second detection unit. The first detection unit is used for detecting whether NS1 is present in an in vitro biological sample to obtain a first detection result, and the second detection unit is used for detecting whether a complex is present in the in vitro biological sample to obtain a second detection result, wherein the complex contains NS1 and thrombin, or contains NS1 and prothrombin. Then, the severity of the dengue virus infection in the individual is evaluated via the first detection result and the second detection result.

In this embodiment, the source of the in vitro biological sample is as mentioned in the Step S01 of the above embodiment.

In this embodiment, the relation of NS1 and thrombin or NS1 and prothrombin is as mentioned in the Step S02 of the above embodiment.

In this embodiment, if the first detection result is that NS1 is present in the in vitro biological sample as well as the second detection result is that the complex is present in the in vitro biological sample, the indicated meaning is as mentioned in the Step S03 of the above embodiment.

The present invention also provides a further embodiment, which relates to a detection kit. Said detection kit comprises the above-mentioned detection device. In addition, the constitution of the detailed components of the detection device, variation form and connection relation with other components are the same as mentioned in the above embodiment, and are not elaborated any further here.

Hereinafter, the present invention will provide a representative example by using serum from patients with dengue virus infection, and illustrate a method for evaluating the severity of the dengue virus infection in an individual according to the present invention in order to supplement to the above description and illustrate that the method of the present invention has high sensitivity and high accuracy at the same time. However, it should be noted that the following descriptions are used to describe the present invention in detail in order to be practiced by a person ordinarily skilled in the art, but not to limit the scope of the present invention.

### Example the Evaluating Method of the Present Invention—Sensitivity Test for Evaluating the Severity of the Dengue Virus Infection in an Individual

### Experimental Materials: Preparation of the Individual Serum Sample

All serum samples donated by the experimental individuals participating in this study are obtained after getting their informed consents. A total of 26 human individuals participated in this study, and all of them were confirmed diagnosed as patients with dengue virus infection by the Centers for Disease Control (CDC, Taiwan). The serum samples are obtained from these patients by collecting blood from the blood vessel of each patient via needles and then centrifuging at 2500 rpm for 25 minutes at room temperature.

### Experimental Methods

This experiment was performed using the SD BIOLINE™ Dengue Duokit (Standard diagnostic Inc.) and following the manufacturer's operating instructions. First, the rapid tests used to perform this experiment were divided into six groups including one experimental group and five comparison groups, that is, Comparison groups 1 to 5. Each rapid test group includes a control line marked as “C”. The rapid tests of Comparison groups 1 and 5 includes test lines marked as “G” and “M”, wherein the test line marked as “G” contained anti-human IgG antibodies as capture antibodies, and the test line marked as “M” contained anti-human IgM antibodies as capture antibodies, and the colloidal gold pad contained membrane protein of dengue virus-colloidal gold. Comparison groups 2, 3, 4 and 5 and Experimental group contained test lines marked as “T”, which contained 1 μL of mouse anti-NS1 monoclonal antibodies (Cat. 12100/12110, Leadgene Biomedical Inc., Taiwan) as capture antibodies, and the colloidal gold pad contained mouse anti-NS1 monoclonal antibody-colloidal gold. Moreover, the colloidal gold pads of Comparison groups 3, 5 and Experimental group further contained 1 μL of sheep anti-thrombin polyclonal antibodies as detecting antibodies in addition to the colloidal gold-labeled mouse anti-NS1 monoclonal antibodies. Subsequently, 80 μL of serum sample was added to a sample pad set in each group of rapid tests and reacted. At the 15th minute of the reaction, the results were interpreted with the naked eyes.

### Experimental Results

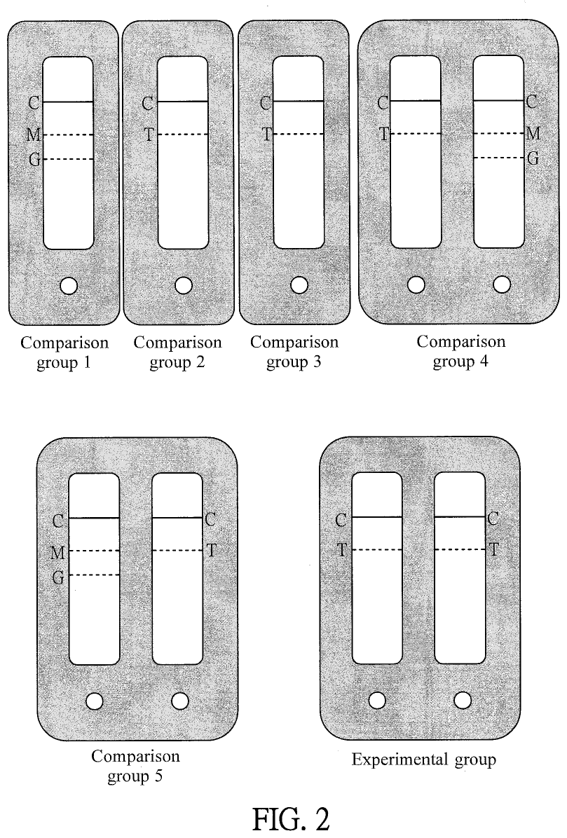

FIG. 2 is a schematic diagram of each group of rapid tests, wherein the meanings of the symbols of the rapid tests have been described in the “Experimental Methods” section of this example, and are not elaborated any further here. Table 1 is a statistical table of the data created after performing the experiment according to the above-mentioned “Experimental Method” and interpreting the results according to the schematic diagram of FIG. 2 (see below).

TABLE 1

| Group No. | Detected Ag or Ab | Sensitivity | |
|---|---|---|---|
| Comparison | Anti-dengue IgG antibody and/or | 9/26 | (34.61%) |
| group 1 | anti-dengue IgM antibody | | |
| Comparison | NS1 | 19/26 | (73.07%) |
| group 2 | | | |
| Comparison | Complex containing NS1 and | 22/26 | (84.61%) |
| group 3 | thrombin | | |
| Comparison | NS1 and anti-dengue IgG antibody | 23/26 | (88.46%) |
| group 4 | and/or anti-dengue IgM antibody | | |
| Comparison | Complex containing NS1 and | 23/26 | (88.46%) |
| group 5 | thrombin and anti-dengue IgG | | |
| | antibody and/or anti-dengue IgM | | |
| | antibody | | |
| Experimental | NS1 and complex containing NS1 | 25/26 | (96.15%) |
| group | and thrombin | | |

As shown in FIG. 2 and Table 1, for Comparison group 1, the anti-dengue IgG antibody and/or anti-dengue IgM antibody were detected in the serum samples from 9 of 26 patients with dengue virus infection, and the sensitivity was 34.61%. For Comparison group 2, NS1 was detected in the serum samples from 19 of 26 patients with dengue virus infection, and the sensitivity is 73.07%. For Comparison group 3, the complex containing NS1 and thrombin was detected in the serum samples from 22 of 26 patients with dengue virus infection, and the sensitivity was 84.61%. For Comparison group 4, NS1 and anti-dengue IgG antibody and/or anti-dengue IgM antibody were detected in the serum samples from 23 of 26 patients with dengue virus infection, and the sensitivity was 88.46%. For Comparison group 5, the complex containing NS1 and thrombin and anti-dengue IgG antibody and/or anti-dengue IgM antibody were detected in the serum samples from 23 of 26 patients with dengue virus infection, and the sensitivity was 88.46%. For Experimental group, NS1 and complex containing NS1 and thrombin were detected in the serum samples from 25 of 26 patients with dengue virus infection, and the sensitivity was 96.15%. The results of this example indicate: the method according to the present invention may accurately detect the dengue virus infection in an individual, and thus have high sensitivity and high accuracy. In particular, the applicant found that the individual belongs to a group B patient with dengue virus infection or a group C patient with dengue virus infection if NS1 and the complex containing NS1 and thrombin are simultaneously present in the serum sample of patient with dengue virus infection (not shown in data). Therefore, the applicant believes that the method according to the present invention can be used to evaluate the severity of dengue virus infection in an individual, and has high sensitivity and high accuracy.

The above description is only exemplary rather than restrictive. Any equivalent modification or change without departing from the spirit and scope of the present invention shall be included in the scope of the appended claims.

What is claimed is:

1. A method for evaluating the severity of the dengue virus infection in an individual, which is executed by means of detecting an in vitro biological sample of an individual, and comprises:
detecting whether a non-structural protein 1 is present in an in vitro biological sample to obtain a first detection result;
detecting whether a complex is present in the in vitro biological sample to obtain a second detection result, wherein the complex contains the non-structural protein 1 and thrombin, or contains the non-structural protein 1 and prothrombin; and
evaluating the severity of the dengue virus infection in the individual via the first detection result and the second detection result.

2. The method of claim 1, wherein the individual belongs to a group B patient with dengue virus infection or a group C patient with dengue virus infection if one of the complex containing the non-structural protein 1 and thrombin and the complex containing the non-structural protein 1 and prothrombin as well as the non-structural protein 1 are simultaneously present in the biological sample.

3. The method of claim 1, wherein the step of detecting whether the non-structural protein 1 is present in the in vitro biological sample is carried out by using an antibody specifically recognizing the non-structural protein 1.

4. The method of claim 1, wherein the step of detecting whether the complex is present in the in vitro biological sample is carried out by using an antibody specifically recognizing the complex.

5. The method of claim 4, wherein the antibody is a monoclonal antibody or a polyclonal antibody.

6. The method of claim 1, wherein the biological sample includes one of followings: blood, urine, saliva and lymph.

7. The method of claim 1, wherein the individual is a mammal.

8. The method of claim 7, wherein the mammal is a human.

9. The method of claim 1, wherein the non-structural protein 1 is a secreted protein or a membrane-associated protein.

10. The method of claim 1, wherein the detection is carried out by using any one of following methodologies: an enzyme linked immunosorbent assay (ELISA), dot blotting, lateral flow assay (LFA), multiplex immunoassay, radioimmunoassay (RIA), immunoradiometric assay (IRMA), fluorescent immunoassay (FIA), chemiluminescent immunoassay and immunonephelometry.

11. A detection device for evaluating the severity of the dengue virus infection in an individual, comprising:
a first detection unit for detecting whether a non-structural protein 1 is present in an in vitro biological sample to obtain a first detection result; and
a second detection unit for detecting whether a complex is present in the in vitro biological sample to obtain a second detection result, wherein the complex contains the non-structural protein 1 and thrombin, or contains the non-structural protein 1 and prothrombin,
wherein the severity of the dengue virus infection in the individual is evaluated via the first detection result and the second detection result.

12. The detection device of claim 11, wherein the biological sample includes one of followings: blood, urine, saliva and lymph.

13. The detection device of claim 11, wherein the individual is a mammal.

14. The detection device of claim 13, wherein the mammal is a human.

15. The detection device of claim 11, wherein the individual belongs to a group B patient with dengue virus infection or a group C patient with dengue virus infection if one of the complex containing the non-structural protein 1 and thrombin and the complex containing the non-structural protein 1 and prothrombin as well as the non-structural protein 1 are simultaneously present in the biological sample.

16. The detection device of claim 11, wherein the non-structural protein 1 is a secreted protein or a membrane-associated protein.

17. A detection kit comprising the detection device of claim 12.

18. The method of claim 3, wherein the antibody is a monoclonal antibody or a polyclonal antibody.

19. The detection kit of claim 17, wherein the individual belongs to a group B patient with dengue virus infection or a group C patient with dengue virus infection if one of the complex containing the non-structural protein 1 and thrombin and the complex containing the non-structural protein 1 and prothrombin as well as the non-structural protein 1 are simultaneously present in the biological sample.

20. The detection kit of claim 17, wherein the non-structural protein 1 is a secreted protein or a membrane-associated protein.
